(19) United States
(12) Patent Application Publication
(10) Pub. No.: US 2026/0002920 A1
(43) Pub. Date: Jan. 1, 2026

(54) Diagnosing A Hydrogen Sensor

(71) Applicant: Schaeffler Technologies AG & Co. KG, Herzogenaurach (DE)

(72) Inventors: Thorsten Knittel, Pentling (DE); Wolfgang Lauerer, Laaber (DE); Andreas Meyer, Zell (DE); Stefan Pesahl, Saal an der Donau (DE)

(73) Assignee: Schaeffler Technologies AG & Co. KG, Herzogenaurach (DE)

(21) Appl. No.: 19/109,819

(22) PCT Filed: Sep. 6, 2023
(86) PCT No.: PCT/EP2023/074376

§ 371 Date: Mar. 7, 2025

(30) Foreign Application Priority Data
Sep. 9, 2022 (DE) 10 2022 209 399.2

(51) Int. Cl. G01N 33/00

(52) U.S. Cl. CPC G01N 33/007; G01N 33/005

(57) ABSTRACT

Various embodiments of the teachings herein include a method of diagnosing a hydrogen sensor having a measurement space, a heater and a temperature sensor, wherein the measurement space is fluidly connected to an environment of the hydrogen sensor by a measurement space opening closed by a gas-permeable and liquid-impermeable membrane. An example includes: changing the temperature in the measurement space using the heater; ascertaining a diagnosis value in the measurement space using the temperature sensor; and determining a diagnosis based at least in part on the diagnosis value.

### CROSS-REFERENCE TO RELATED APPLICATIONS

This application is a U.S. National Stage Application of International Application No. PCT/EP2023/074376 filed Sep. 6, 2023, which designates the United States of America, and claims priority to DE Application No. 10 2022 209 399.2 filed Sep. 9, 2022, the contents of which are hereby incorporated by reference in their entirety.

### TECHNICAL FIELD

The present disclosure relates to hydrogen sensors. Various embodiments of the teachings herein include systems and methods for diagnosing a hydrogen sensor and hydrogen sensors.

### BACKGROUND

Hydrogen sensors for ascertaining a hydrogen concentration of a gas generally have a measurement space that offers protection from the flow of gas. Access to this measurement space is generally closed by a gas-permeable and liquid-impermeable membrane in order to prevent the ingress of liquid or foreign bodies into the measurement space. Deposits of foreign bodies or accumulation of liquid occur in an undesirable manner at this membrane, which distorts measurement in the hydrogen concentration of the gas.

Document U.S. Pat. No. 9,702,836 B2 relates to a sensor having an error recognition function.

### SUMMARY

Teachings of the present disclosure include systems and methods notable for ease of implementation. For example, some embodiments include a method of diagnosing a hydrogen sensor (4), wherein the hydrogen sensor (4) comprises a measurement space, a heater and a temperature sensor, wherein the measurement space is fluidically connected to the environment of the hydrogen sensor by means of a measurement space opening, wherein the measurement space opening is closed by means of a membrane (5), wherein the membrane (5) is gas-permeable and liquid-impermeable, the method comprising: changing the temperature (7) in the measurement space by means of the heater, ascertaining (8) a diagnosis value in the measurement space by means of the temperature sensor, and determining (11, 12) a diagnosis depending on the diagnosis value.

In some embodiments, the change in temperature (7) comprises operation of the heater with a constant heating output, heating by means of the heater by an increase in the heating output, cooling owing to deactivation and/or lowering of the heating output of the heater.

In some embodiments, the ascertained diagnosis value comprises a temperature, a rate of temperature change, a progression of temperature change and/or a time value.

In some embodiments, the method comprises the method step of comparing (10) the ascertained diagnosis value with a recorded diagnosis value (9).

In some embodiments, the recorded diagnosis value (9) is of the same type as the ascertained diagnosis value.

In some embodiments, a diagnosis is determined when the ascertained diagnosis value attains, goes below and/or goes above the recorded diagnosis value (9).

In some embodiments, the change in temperature (7) in the measurement space and the ascertaining (8) of the diagnosis value are conducted simultaneously, after a time delay or in direct succession.

In some embodiments, the change in temperature (7) in the measurement space and the ascertaining (8) of the diagnosis value are started and/or ended simultaneously, after a time delay or in direct succession.

In some embodiments, the change in temperature (7) in the measurement space and/or the ascertaining (8) of the diagnosis value is started when the temperature in the measurement space goes below or above a particular temperature, and/or in that the change in temperature (7) in the measurement space and/or the ascertaining (8) of the diagnosis value is ended when the temperature in the measurement space goes below or above particular temperature.

In some embodiments, the determining (11, 12) of the diagnosis comprises the determining of a blockage of the membrane with a foreign body or a liquid.

In some embodiments, the method is a computer-implemented method.

As another example, some embodiments include a hydrogen sensor (4) comprising a measurement space, a heater and a temperature sensor, wherein the measurement space is fluidically connected to the environment of the hydrogen sensor (4) by means of a measurement space opening, wherein the measurement space opening is closed by means of a membrane (5), wherein the membrane (5) is gas-permeable and liquid-impermeable, and comprising means adapted to execute one or more of the methods described herein.

As another example, some embodiments include a computer program (16) comprising commands that cause a device to execute one or more of the methods described herein.

As another example, some embodiments include a computer-readable medium (15) on which a computer program (16) as described herein is stored.

### BRIEF DESCRIPTION OF THE DRAWINGS

The teachings herein are elucidated in detail hereinafter by working examples with reference to the drawings. The drawings show:

FIG. 1 a motor vehicle incorporating teachings of the present disclosure;

FIG. 2 a conduit comprising an example hydrogen sensor incorporating teachings of the present disclosure; and

FIG. 3 a flow diagram of an example method incorporating teachings of the present disclosure.

### DETAILED DESCRIPTION

As an example, some embodiments of the teachings herein include a method of diagnosing a hydrogen sensor, wherein the hydrogen sensor comprises a measurement space, a heater and a temperature sensor, wherein the measurement space is fluidically connected to the environment of the hydrogen sensor by means of a measurement space opening, wherein the measurement space opening is closed by means of a membrane, wherein the membrane is gas-permeable and liquid-impermeable, comprising: changing the temperature in the measurement space by means of the heater, ascertaining a diagnosis value in the measurement space by means of the temperature sensor, and determining a diagnosis depending on the diagnosis value.

In this way, it is possible to perform a diagnosis of the hydrogen sensor without having to provide additional sensor elements, since existing means are utilized to ascertain the hydrogen concentration in a gas by means of the hydrogen sensor for diagnosis. The determining of the diagnosis may include the determining of the diagnosis of the hydrogen sensor, the membrane thereof, and/or the measurement space opening thereof.

In some embodiments, the heater and the temperature sensor are disposed in the measurement space. This enables faster heating and more exact measurement.

In some embodiments, the hydrogen sensor is a thermal sensor in particular for ascertaining a hydrogen concentration in a gas.

In some embodiments, the hydrogen sensor is a thermal sensor that utilizes the much higher heat capacity of hydrogen compared to other constituents in the gas to be analyzed in order to ascertain the hydrogen concentration in the gas to be analyzed.

In some embodiments, the hydrogen sensor comprises a sensor housing which bounds the measurement space and/or comprises the measurement space opening, e.g. at the surface of the sensor housing.

In some embodiments, the change in temperature comprises heating or cooling. Heating, i.e. warming, is possible by operation of the heater, while cooling is possible by switching off the heater if it was in operation beforehand.

In some embodiments, the determining is a measurement.

In some embodiments, the method is conducted prior to ascertaining the hydrogen concentration of a gas. In this way, it is possible to verify the functioning of the sensor prior to use thereof.

In some embodiments, the diagnosis value is ascertained in reaction to the change in temperature in the measurement space.

In some embodiments, the diagnosis is determined in reaction to the determination of the diagnosis value.

In some embodiments, the change in temperature comprises operation of the heater with a constant heating output, heating by means of the heater by an increase in the heating output, cooling owing to deactivation and/or lowering of the heating output of the heater.

In some embodiments, the ascertained diagnosis value comprises a temperature, a rate of temperature change, a progression of temperature change and/or a time value. The temperature, e.g. the temperature in the measurement space, can be ascertained or measured, for example, after a particular time interval. The rate of temperature change means the change in temperature per unit time. The temperature progression means the progression of temperature during a time interval. If solely a temperature is used as diagnosis value, this is particularly rapid, while the rate of temperature change or the progression of temperature change permit particularly exact conclusions. In the case of the time value, it is possible to use, for example, the time taken to get from a starting temperature to a target temperature. When the membrane or the measurement space opening is blocked by a foreign body or a liquid, the measurement space will heat up more quickly, i.e. within a shorter time, than with a clear membrane and clear measurement space opening.

In some embodiments, the aforementioned different diagnosis values that are possible are relative or absolute values.

In some embodiments, before the start of the method or before commencement of the change in temperature, the starting temperature in the measurement space is ascertained and is taken into account in the determining of the diagnosis or in the ascertaining of the diagnosis value.

In some embodiments, the method comprises the method step of comparing the ascertained diagnosis value with a recorded diagnosis value. The recorded diagnosis value is, for example, a diagnosis value or comparison value that has been ascertained experimentally beforehand. This may alternatively be a diagnosis value or comparison value ascertained beforehand by means of the method of the invention. In other words, this is a historic ascertained diagnosis value or comparison value from the past, which is utilized as a recorded diagnosis value or comparison value. The comparison may be made after the ascertaining and/or before the determining of the diagnosis.

In some embodiments, the recorded diagnosis value is of the same type as the ascertained diagnosis value. For example, an ascertained progression of temperature change is compared with a recorded progression of temperature change, or an ascertained time value with a recorded time value.

In some embodiments, a diagnosis is determined when the ascertained diagnosis value attains, goes below and/or goes above the recorded diagnosis value. It is possible by virtue of these variants to address different use conditions of the sensor.

In some embodiments, the change in temperature in the measurement space and the ascertaining of the diagnosis value are conducted simultaneously, after a time delay or in direct succession. In this way, it is possible to vary the mutual influence between the change in temperature and the ascertaining.

In some embodiments, the change in temperature in the measurement space and the ascertaining of the diagnosis value are started and/or ended simultaneously, after a time delay or in direct succession. In this way too, it is possible to vary the mutual influence between the change in temperature and the ascertaining.

In some embodiments, the change in temperature in the measurement space and/or the ascertaining of the diagnosis value is started when the temperature in the measurement space goes below or above a particular temperature, and/or in that the change in temperature in the measurement space and/or the ascertaining of the diagnosis value is ended when the temperature in the measurement space goes below or above a particular temperature.

In some embodiments, the determining of the diagnosis comprises the determining of a blockage of the membrane with a foreign body or a liquid. These two determinations are two typical causes that influence the diagnosis values to be ascertained. Therefore, depending on the diagnosis value, it is possible to conclude one of these two states, especially when the ascertained diagnosis value is compared with a recorded diagnosis value, where the recorded diagnosis value corresponds to one of the two states.

In some embodiments, in the case of determining a blockage of the membrane with a foreign body or a liquid, a measurement by means of the hydrogen sensor is refused or is not possible. This may be impossible if it is determined that the membrane is blocked with a foreign body or a liquid. For this purpose, it is appropriate to repeat the method several times, in particular at time intervals.

In some embodiments, it is conceivable to label, identify or store measurements relating to the concentration of hydrogen in the gas to be analyzed that are ascertained by means of the hydrogen sensor after determination of a blockage of the membrane with a foreign body or a liquid as impermissible or erroneous. In this way, it is possible to prevent utilization of the measurements for open-loop or closed-loop control of a system, especially a fuel cell system.

In some embodiments, the heating output of the heater is increased in order to remove the foreign body or the liquid. Alternatively or additionally thereto, it is conceivable to briefly increase the flow rate of the gas to be analyzed in order to remove the foreign body or the liquid.

In some embodiments, in the case that a diagnosis is determined that the membrane or the measurement space opening is free of a liquid or a foreign body, the method is ended.

In some embodiments, the method is a computer-implemented method.

As another example, some embodiments of the teachings herein include a hydrogen sensor comprising a measurement space, a heater and a temperature sensor, wherein the measurement space is fluidically connected to the environment of the hydrogen sensor by means of a measurement space opening, wherein the measurement space opening is closed by means of a membrane, wherein the membrane is gas-permeable and liquid-impermeable, and comprising means adapted to execute one or more of the methods described herein.

In some embodiments, the hydrogen sensor is also coupled to a control device which comprises the recorded diagnosis values. In some embodiments, the control device comprises an analysis unit that performs the determining of the diagnosis and/or the comparison of the diagnosis value ascertained with a recorded diagnosis value.

In some embodiments, the heater and the temperature sensor are disposed in the measurement space. This enables faster heating and more exact measurement.

In some embodiments, the hydrogen sensor is a sensor for ascertaining a hydrogen concentration in a gas. Such sensors are used in exhaust gas conduits of fuel cell systems.

In some embodiments, the hydrogen sensor comprises a sensor housing that bounds the measurement space.

Some embodiments include a computer program including commands that cause the hydrogen sensor to execute the one or more of the methods described herein. A computer-readable medium may store a computer program as described herein.

In some embodiments, the computer-readable medium is part of the control device, for example as a permanent or temporary storage medium.

Technical developments of the present teachings are described in the description of figures that follows.

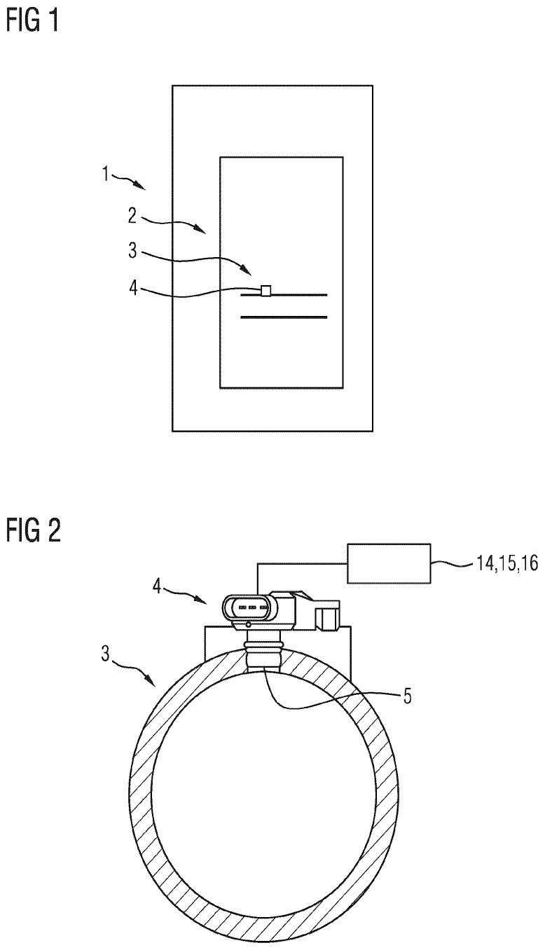

FIG. 1 shows a motor vehicle 1 with electrical drive and a fuel cell system 2. The fuel cell system 2 comprises a conduit 3 in the form of an exhaust gas conduit of the fuel cell system 2 and in which the hydrogen concentration can be ascertained by means of a hydrogen sensor 4 incorporating teachings of the present disclosure.

FIG. 2 shows the conduit 3 and the hydrogen sensor 4 from FIG. 1. The hydrogen sensor 4 is secured in an opening of the conduit wall of the conduit 3 and comprises a measurement space fluidically connected to the conduit 3 by means of a measurement space opening. The measurement space opening is closed by means of a membrane 5 which is gas-permeable and liquid-impermeable. A heater and a temperature sensor are disposed in the measurement space of the hydrogen sensor 4. By means of the high heat capacity of hydrogen compared to the other constituents of the gas to be analyzed, it is possible by means of the heater and the temperature sensor to ascertain the concentration of hydrogen in the gas to be analyzed. In other words, the hydrogen sensor 4 is a thermal gas sensor. The hydrogen sensor 4 is coupled to a control device 14 incorporating a computer-readable medium 15 in the form of a permanent storage medium. Stored on the computer-readable medium 15 is a computer program 16 comprising commands that cause the hydrogen sensor 4 and/or an evaluation unit integrated into the control device 14 to execute the method steps of the method of the invention.

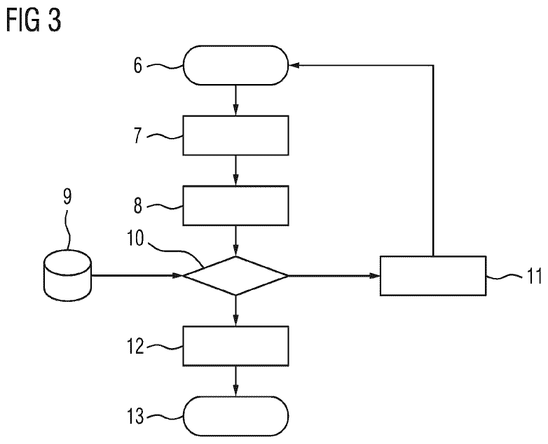

FIG. 3 shows a flow diagram of an example method incorporating teachings of the present disclosure, as employed in the devices of FIGS. 1 and 2. First of all, the method is started 6 before a change in temperature 7 is performed in the measurement space of the hydrogen sensor by means of the heater. The change in temperature 7 is a heating operation that increases the temperature in the measurement space. Simultaneously with the changes in temperature 7, a diagnosis value is ascertained 8 by means of the temperature sensor. The diagnosis value ascertained is the time taken for a certain temperature to be attained within the measurement space. This is followed by a comparison 10 of this diagnosis value with a recorded diagnosis value 9. When the ascertained diagnosis value is below the recorded diagnosis value 9, this means that the temperature in the measurement space has increased more quickly than usual, since the recorded diagnosis value 9 corresponds to a time value that can be ascertained when the membrane of the hydrogen sensor is blocked neither by a liquid nor by a foreign body. If the ascertained diagnosis value is below the recorded diagnosis value 9, the determination 11 is therefore made that the membrane or the measurement space opening is blocked by a foreign body or a liquid.

Thereafter, it is possible to restart the method at a later juncture, in order to determine whether this diagnosis is still correct. In addition, it is conceivable to continue to operate the heater, and to remove the liquid or the foreign body. In addition, it is conceivable to initialize a cleaning method for cleaning of the membrane or the measurement space opening. It is also conceivable that measurements relating to the concentration of hydrogen in the gas to be analyzed are stored as erroneous values and therefore not used for further methods. If the ascertained diagnosis value corresponds at least to the recorded diagnosis value 9, the determination 12 is made that the membrane or the measurement space opening is free of a liquid or a foreign body. Thereafter, the method is ended 13, as a result of which it is possible thereafter to initialize a measurement method to ascertain the concentration of hydrogen in the gas to be analyzed.

The different features of the individual working examples may also be combined with one another.

In particular, the working examples of FIGS. 1 to 3 do not have any limiting character and serve to clarify the concept of the disclosure without limiting its scope.

### LIST OF REFERENCE NUMERALS

1 motor vehicle
2 fuel cell system
3 conduit
4 hydrogen sensor
5 membrane
6 start
7 change in temperature
8 ascertainment
9 recorded diagnosis value
10 comparison
11 determination
12 determination
13 end
14 control device
15 computer-readable medium
16 computer program

What is claimed is:

1. A method of diagnosing a hydrogen sensor having a measurement space, a heater and a temperature sensor,
wherein the measurement space is fluid connected to an environment of the hydrogen sensor by a measurement space opening closed by a gas-permeable and liquid-impermeable membrane,
the method comprising:
changing the temperature in the measurement space using the heater;
ascertaining a diagnosis value in the measurement space using the temperature sensor; and
determining a diagnosis based at least in part on the diagnosis value.

2. (canceled)

3. The method as claimed in claim 1, wherein the ascertained diagnosis value comprises a temperature, a rate of temperature change, a progression of temperature change, and/or a time value.

4. The method as claimed in claim 1, further comprising comparing the ascertained diagnosis value with a recorded diagnosis value.

5. The method as claimed in claim 4, wherein the recorded diagnosis value is of a matching type with the ascertained diagnosis value.

6. The method as claimed in claim 1, wherein a diagnosis is determined when the ascertained diagnosis value attains, goes below, and/or goes above the recorded diagnosis value.

7. The method as claimed in claim 1, wherein changing the temperature in the measurement space and ascertaining the diagnosis value are conducted simultaneously, after a time delay, or in direct succession.

8. The method as claimed in claim 1, wherein changing the temperature in the measurement space and ascertaining the diagnosis value are started and/or ended simultaneously, after a time delay, or in direct succession.

9. The method as claimed in claim 1, wherein changing the temperature in the measurement space and/or ascertaining the diagnosis value is started when the temperature in the measurement space goes below or above a particular temperature, and/or ended when the temperature in the measurement space goes below or above a particular temperature.

10. The method as claimed in claim 1, wherein determining the diagnosis comprises determining a blockage of the membrane by a foreign body or a liquid.

11. (canceled)

12. A hydrogen sensor comprising:
a measurement space;
a heater;
a temperature sensor;
wherein the measurement space is fluid connected to an environment of the hydrogen sensor by a measurement space opening;
a membrane closing the measurement space opening;
wherein the membrane is gas-permeable and liquid-impermeable; and
a controller operable to change the temperature in the measurement space using the heater, ascertain a diagnosis value in the measurement space using the temperature sensor, and determine a diagnosis based at least in part on the diagnosis value.

13-14. (canceled)
